(19) United States
(12) Patent Application Publication
(10) Pub. No.: US 2012/0010468 A1
(43) Pub. Date: Jan. 12, 2012

(54) SYSTEM OF DISPOSABLE CONTOURED SLEEVES FOR FLEXIBLE ENDOSCOPES

(76) Inventor: Adel Afridi, Danvers, MA (US)

(21) Appl. No.: 13/177,528

(22) Filed: Jul. 6, 2011

Related U.S. Application Data
(60) Provisional application No. 61/361,569, filed on Jul. 6, 2010.

(51) Int. Cl. A61B 1/00

(52) U.S. Cl. 600/121

(57) ABSTRACT

A system of for labeling and organizing de-contaminated and cleaned flexible endoscopes with coded one-use disposable sheaths. The sheaths are specifically contoured and coded for specific types of endoscopes. The endoscopes can only be removed from sheaths by tearing, slicing, cutting or otherwise breaking the sheaths open. This product system reduces the risk of patient to patient cross-contamination by labeling cleaned endoscopes for easy identification by medical care professionals.

### CROSS REFERENCE TO RELATED APPLICATION

This application claims the benefit of U.S. Provisional Patent Application No. 61/361,569 filed on Jul. 6, 2010, the contents of which are incorporated herein by reference.

### FIELD OF THE INVENTION

The field of the invention relates to a system for labeling de-contaminated and cleaned flexible endoscopes. This product system reduces the risk of patient to patient cross-contamination by labeling cleaned endoscopes for easy identification by medical care professionals.

### BACKGROUND OF INVENTION

Flexible endoscopes are a wide array of surgical instruments used by medical care providers to conduct various medical procedures, including observation, diagnosis, and surgery, within the tight confines of internal body cavities in a minimally invasive manner, i.e., through artificial or natural body orifices.

Virtually all flexible endoscopes have insertion tubing which is a flexible elongated tubular member on their distal ends. For the purposes of this application, and, as known to those skilled in the art, “distal” shall refer to the direction away from the medical care provider, near the anatomical site, and “proximal” shall refer to the direction near the medical care provider, away from the anatomical site.

Often this insertion tubing contains a tool for observation, diagnosis or cutting on the distal tip, such as a miniature video camera, a fiber optic bundle (which carries illumination forward to illuminate an anatomical site within the patient's body cavities), surgical blades, surgical clip applicators or emulsifiers.

Most flexible endoscopes also have one or multiple inner channels, usually used to carry tools for providing fluids to irrigate anatomical sites, vacuum components to provide suction to anatomical sites, or retrieval tools, such as claws, to obtain tissue from anatomical sites for biopsy.

To prevent cross contamination between patients, all flexible endoscopes have to be properly decontaminated and disinfected after each use. Typically, once the endoscopes are processed i.e., cleaned and decontaminated, they are kept in a separate area to distinguish them from dirty or used endoscopes.

However, in crowded portions of hospitals, such as emergency rooms, clean and dirty endoscopes may be hanging side by side. The only labeling system used to distinguish between clean (ready for use) from dirty (already used) endoscopes is often just different colored tags placed on the endoscopes. These tags are placed on the clean endoscopes to indicate that an endoscope has been processed and is ready for use. Such tags are often cumbersome to remove, not clearly noticeable, and even if left in place do not interfere with use of the endoscope. Thus, in emergency situations, the colored tags could be left in place even after the endoscope has been used and the next user may unknowingly use a contaminated endoscope.

Accordingly, a need exists for a cheap, easy to manufacture and an easy to use system endoscope identification that distinguishes between clean and used endoscopes. This system should be easy and quick to install over clean endoscopes. The system should be flexible enough to fit the multiple types of endoscopes used by medical care providers. The system should be easy to remove from cleaned endoscopes. The system should prevent a medical care provider from using the endoscope without removing the identification, thus facilitating identification between a dirty endoscope and a clean endoscope without interfering with endoscope usage during emergencies.

### SUMMARY OF THE INVENTION

There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

The subject invention discloses a system for organizing de-contaminated and clean endoscopes, comprising: cleaning and de-contaminating an endoscope with an elongated shaft; selecting a disposable elongated sheath based on the type of endoscope to be enclosed from a plurality of disposable elongated sheaths, wherein each sheath from the plurality of sheaths closely fits a different type of endoscope, further wherein each sheath comprises indicia to indicate the type of endoscope that the sheath closely fits, wherein each sheath comprises a thin-walled hollow tubular body composed of a flexible plastic, a proximal end with an proximal opening, a distal end with a distal opening, further wherein each sheath comprises a generally circular cross-section and a substantially frustoconical configuration such that the proximal opening has a larger diameter than the distal opening; placing the endoscope into the selected sheath by inserting the distal end of the endoscope through the proximal opening, wherein the proximal end of the sheath is adapted to allow the endoscope to pass through it; passing the distal end of the endoscope through the hollow tubular body; passing a small portion of the distal end of the endoscope through the distal opening such that the sheath closely fits around that endoscope, wherein the distal opening of the sheath is adapted to only allow the small portion of the distal end of the endoscope to pass through; folding a flap that is hingedly attached to the sheath proximate to the proximal opening over the proximal opening, wherein the flap has an adhesive strip; attaching the folded flap to the body with the adhesive strip to maintain the enclosed endoscope in an axially aligned position within the sheath, wherein the enclosed endoscope can only be removed from the sheath by breaking the thin wall of the sheath; placing the sheathed endoscope into a location for clean and de-contaminated endoscopes designated by a user; removing the sheathed endoscope for use by breaking the thin wall of the sheath and removing the enclosed endoscope; and discarding the used sheath.

Another embodiment of the subject invention is a system for organizing de-contaminated and clean endoscopes, comprising: cleaning and de-contaminating an endoscope with an elongated shaft; selecting a non-reusable elongated sleeve based on the type of endoscope to be enclosed from a plurality of disposable elongated sleeves, wherein each sleeve comprises indicia to indicate the type of endoscope enclosed, further wherein each sleeve comprises a thin-walled hollow tubular body composed of a flexible plastic, a proximal end with an proximal opening, a distal end with a distal opening, further wherein each sleeve comprises a generally circular cross-section and a substantially frustoconical configuration such that the proximal opening has a larger diameter than the distal opening; placing the endoscope into the sleeve by inserting the distal end of the endoscope through the proximal opening, wherein the proximal end of the sleeve is adapted to allow the endoscope to pass through it; passing the distal end of the endoscope through the hollow tubular body; passing a small portion of the distal end of the endoscope through the distal opening such that the sleeve closely fits around that endoscope, wherein the distal opening of the sleeve is adapted to only allow the small portion of the distal end of the endoscope to pass through; folding a flap that is hingedly attached to the sleeve proximate to the proximal opening over the proximal opening, wherein the flap has an adhesive strip; attaching the folded flap to the body with the adhesive strip to maintain the enclosed endoscope in an axially aligned position within the sleeve, wherein the enclosed endoscope can only be removed from the sleeve by tearing, slicing, cutting or breaking the thin wall of the sleeve; placing the sleeved endoscope into a location for clean and de-contaminated endoscopes designated by a user; removing the sleeved endoscope for use by tearing, slicing, cutting or breaking the thin wall the sleeve and removing the enclosed endoscope; and discarding the used sleeve.

In a further embodiment of the subject invention, the indicia to indicate the type of endoscope enclosed may be a color-code.

In an additional embodiment of the subject invention, the indicia to indicate the type of endoscope enclosed may be a written label.

In another embodiment of the subject invention, the walls of the body may have a thickness less than 2 millimeters.

In one embodiment of the subject invention, the flap may comprise an exterior writable surface. In another embodiment of the subject invention, the sheath or sleeve may comprise an exterior writable surface.

In another embodiment of the subject invention, the sheath or sleeve may comprise a substantially U-shaped opening on the flap, wherein the U-shaped opening is adapted to allow an external adaptor on the endoscope to protrude from underneath the closed flap.

In a further embodiment of the subject invention, the sleeve or sheath may comprise an substantially U-shaped flap attached to an edge of the flap, wherein the U-shaped flap is adapted to allow an external adaptor on the endoscope to protrude from underneath the closed flap.

In another embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

An additional embodiment of the subject invention is a system for organizing de-contaminated and clean endoscopes, comprising: cleaning and de-contaminating an endoscope with an elongated shaft; selecting a disposable single-use elongated protective covering based on the type of endoscope to be enclosed from a plurality of disposable elongated protective coverings, wherein each protective covering comprises indicia to indicate the type of endoscope enclosed, further wherein each protective covering comprises a thin-walled hollow tubular body composed of an elastic plastic, a proximal end with an proximal opening, a distal end with a distal opening, further wherein each protective covering comprises a generally circular cross-section and a substantially frustoconical configuration such that the proximal opening has a larger diameter than the distal opening; placing the endoscope into the protective covering by inserting the distal end of the endoscope through the proximal opening, wherein the proximal end of the protective covering is adapted to allow the endoscope to pass through it; passing the distal end of the endoscope through the hollow tubular body; passing a small portion of the distal end of the endoscope through the distal opening such that the protective covering closely fits around that endoscope, wherein the distal opening of the protective covering is adapted to only allow the small portion of the distal end of the endoscope to pass through; folding a flap that is hingedly attached to the protective covering proximate to the proximal opening over the proximal opening, wherein the flap has an adhesive strip; attaching the folded flap to the body with the adhesive strip to maintain the enclosed endoscope in an axially aligned position within the protective covering, wherein the enclosed endoscope can only be removed from the protective covering by tearing, slicing, cutting or breaking the thin wall of the protective covering; placing the protective covered endoscope into a location for clean and de-contaminated endoscopes designated by a user; removing the protective covered endoscope for use by tearing, slicing, cutting or breaking the thin wall of the protective covering and removing the enclosed endoscope; and discarding the used protective covering.

In another embodiment of the subject invention, the indicia to indicate the type of endoscope enclosed may be a color-code.

In a further embodiment of the subject invention, the indicia to indicate the type of endoscope enclosed may be a written label.

In an additional embodiment of the subject invention, the walls of the body may have a thickness less than 2 millimeters.

In another embodiment of the subject invention, the flap may comprise an exterior writable surface. In another embodiment of the subject invention, the protective covering may comprise an exterior writable surface.

In a further embodiment of the subject invention, the protective covering may comprise a substantially U-shaped opening on the flap, wherein the U-shaped opening is adapted to allow an external adaptor on the endoscope to protrude from underneath the closed flap.

In another embodiment of the subject invention, the protective covering may comprise an substantially U-shaped flap attached to an edge of the flap, wherein the U-shaped flap is adapted to allow an external adaptor on the endoscope to protrude from underneath the closed flap.

In an additional embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

The subject invention also discloses a disposable elongated sheath adapted for covering a single endoscope with an elongated shaft, said sheath comprising: a thin-walled hollow tubular body composed of a flexible plastic, wherein the body comprises a proximal end with an proximal opening and a distal end with a distal opening, further wherein the body comprises a generally circular cross-section and a substantially frustoconical configuration wherein the proximal opening has a larger diameter than the distal opening; wherein the sheath is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the sheath closely fits around that endoscope; a flap hingedly attached to the sheath proximate to the proximal opening on the sheath, wherein the flap has an adhesive strip, such that the flap folds over the proximal opening and attaches to the body with the adhesive strip to maintain the enclosed endoscope in an axially aligned position within the sheath, wherein the enclosed endoscope can only be removed from the sheath by tearing, cutting, or slicing the sheath open; and a pre-formed tearable perforation on the body to remove the enclosed endoscope.

Another embodiment of the subject invention is a non-reusable elongated sheath sized to accommodate a single endoscope with an elongated shaft, said sheath comprising: a thin-walled hollow tubular body with a longitudinal axis composed of an elastic plastic, wherein the body comprises a generally circular cross-section; a proximal end on the body with an proximal opening; a distal end on the body with a distal opening, wherein the proximal opening has a larger diameter than the distal opening such that the body comprises a substantially frustoconical configuration; wherein the sheath is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the sheath closely fits around that endoscope; a flap hingedly attached to the sheath proximate to the proximal opening on the sheath, wherein the flap has an adhesive strip, such that the flap folds over the proximal opening and attaches to the body with the adhesive strip to maintain the enclosed endoscope in an axially aligned position within the sheath, wherein the enclosed endoscope can only be removed from the sheath by tearing, cutting, or slicing the sheath open; and a pre-formed tearable perforation on the body to remove the enclosed endoscope.

In a further embodiment of the subject invention, the walls of the body may have a thickness less than 2 millimeters.

In another embodiment of the subject invention, the flap may comprise an exterior writable surface. In a further embodiment of the subject invention, the sheath may comprise an exterior writable surface.

In an additional embodiment of the subject invention, the sheath may comprise a substantially U-shaped opening on the flap, wherein the U-shaped opening is adapted to allow an external adaptor on the endoscope to protrude from underneath the closed flap.

In another embodiment of the subject invention, the sheath may comprise an substantially U-shaped flap attached to an edge of the flap, wherein the U-shaped flap is adapted to allow an external adaptor on the endoscope to protrude from underneath the closed flap.

In an additional embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

Another embodiment of the subject invention is a disposable single-use elongated sheath sized to protectively cover a single endoscope with an elongated shaft, said sheath comprising: elongated tubular sidewalls composed of a flexible plastic; a proximal end with an proximal opening; a distal end with a distal opening, wherein the sheath comprises a generally circular cross-section and a substantially frustoconical configuration wherein the proximal opening has a larger diameter than the distal opening; wherein the proximal opening is adapted to receive the entire endoscope and the distal opening is adapted to receive only a small portion of the distal end of the endoscope such that the sheath closely fits around that endoscope; a flap hingedly attached to an edge of the proximal opening on the sheath, wherein the flap has an adhesive strip, such that the flap folds over the proximal opening and attaches to the body with the adhesive strip to maintain the enclosed endoscope in an axially aligned position within the sheath, wherein the enclosed endoscope can only be removed from the sheath by tearing, cutting, or slicing the sheath open; and a pre-formed tearable perforation on the body for removing the enclosed endoscope.

In a further embodiment of the subject invention, the tubular sidewalls may have a thickness less than 2 millimeters.

In another embodiment of the subject invention, the flap may comprise an exterior writable surface. In another embodiment of the subject invention, the sheath may comprise an exterior writable surface.

In a further embodiment of the subject invention, the sheath may comprise a substantially U-shaped opening on the flap, wherein the U-shaped opening is adapted to allow an external adaptor on the endoscope to protrude from underneath the closed flap.

In another embodiment of the subject invention, the sheath may comprise an substantially U-shaped flap attached to an edge of the flap, wherein the U-shaped flap is adapted to allow an external adaptor on the endoscope to protrude from underneath the closed flap.

In an additional embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

Another embodiment of the subject invention is a non-reusable elongated sheath sized to accommodate a single endoscope with an elongated shaft, said sheath comprising: a thin-walled hollow tubular body with a longitudinal axis composed of an elastic plastic, wherein the body comprises a generally circular cross-section; a proximal end on the body with an proximal opening; a distal end on the body with a distal opening, wherein the proximal opening has a larger diameter than the distal opening such that the body comprises a substantially cylindrical shape that is tapered; wherein the sheath is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the sheath closely fits around that endoscope; a substantially flat extension hingedly attached to an edge of the proximal opening on the sheath, wherein the extension has an adhesive strip, such that the extension folds over the proximal opening and attaches to the body with the adhesive strip to maintain the enclosed endoscope in an axially aligned position within the sheath, wherein the enclosed endoscope can only be removed from the sheath by tearing, cutting, or slicing the sheath open; and a pre-formed tearable perforation on the body to remove the enclosed endoscope.

In a further embodiment of the subject invention, the walls of the body may have a thickness less than 2 millimeters.

In another embodiment of the subject invention, the substantially flat extension may comprise an exterior writable surface. In another embodiment of the subject invention, the sheath may comprise an exterior writable surface.

In a further embodiment of the subject invention, the sheath may comprise a substantially U-shaped opening on the substantially flat extension, wherein the U-shaped opening is adapted to allow an external adaptor on the endoscope to protrude from underneath the closed substantially flat extension.

In an additional embodiment of the subject invention, the sheath may comprise an substantially U-shaped flap attached to an edge of the substantially flat extension, wherein the U-shaped flap is adapted to allow an external adaptor on the endoscope to protrude from underneath the closed substantially flat extension.

In another embodiment of the subject invention, the substantially flat extension may fold over the proximal opening and attach to the body with the adhesive strip such that any external adaptors connected to the enclosed endoscope may freely protrude from underneath the folded flat extension.

In a further embodiment of the subject invention, the adhesive strip may have a protective peel off strip that is removed before attaching the substantially flat extension to the body.

The subject invention further discloses a disposable elongated sleeve adapted for covering a single endoscope with an elongated shaft, said sleeve comprising: a thin-walled hollow tubular body composed of a flexible plastic, wherein the body comprises a proximal end with an proximal opening and a distal end with a distal opening, further wherein the body comprises a generally circular cross-section and a substantially frustoconical configuration wherein the proximal opening has a larger diameter than the distal opening; wherein the sleeve is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the sleeve closely fits around that endoscope; a flap hingedly attached to the sleeve proximate to the proximal opening on the sleeve, wherein the flap has an adhesive strip, such that the flap folds over the proximal opening and attaches to the body with the adhesive strip to maintain the enclosed endoscope in an axially aligned position within the sleeve, wherein the enclosed endoscope can only be removed from the sleeve by tearing, cutting, or slicing the sleeve open; and a pre-formed tearable perforation on the body to remove the enclosed endoscope.

Another embodiment of the subject invention is a non-reusable elongated sleeve sized to accommodate a single endoscope with an elongated shaft, said sleeve comprising: a thin-walled hollow tubular body with a longitudinal axis composed of an elastic plastic, wherein the body comprises a generally circular cross-section; a proximal end on the body with an proximal opening; a distal end on the body with a distal opening, wherein the proximal opening has a larger diameter than the distal opening such that the body comprises a substantially frustoconical configuration; wherein the sleeve is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the sleeve closely fits around that endoscope; a flap hingedly attached proximate to the proximal opening on the sleeve, wherein the flap has an adhesive strip, such that the flap folds over the proximal opening and attaches to the body with the adhesive strip to maintain the enclosed endoscope in an axially aligned position within the sleeve, wherein the enclosed endoscope can only be removed from the sleeve by tearing, cutting, or slicing the sleeve open; and a pre-formed tearable perforation on the body to remove the enclosed endoscope.

In a further embodiment of the subject invention, the walls of the body may have a thickness less than 2 millimeters.

In an additional embodiment of the subject invention, the flap may comprise an exterior writable surface. In another embodiment of the subject invention, the sleeve may comprise an exterior writable surface.

In another embodiment of the subject invention, the sleeve may comprise a substantially U-shaped opening on the flap, wherein the U-shaped opening is adapted to allow an external adaptor on the endoscope to protrude from underneath the closed flap.

In a further embodiment of the subject invention, the sleeve may comprise an substantially U-shaped flap attached to an edge of the flap, wherein the U-shaped flap is adapted to allow an external adaptor on the endoscope to protrude from underneath the closed flap.

In another embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

Another embodiment of the subject invention is a disposable single-use elongated sleeve sized to protectively cover a single endoscope with an elongated shaft, said sleeve comprising: elongated tubular sidewalls composed of a flexible plastic; a proximal end with an proximal opening; a distal end with a distal opening, wherein the sleeve comprises a generally circular cross-section and a substantially frustoconical configuration wherein the proximal opening has a larger diameter than the distal opening; wherein the proximal opening is adapted to receive the entire endoscope and the distal opening is adapted to receive only a small portion of the distal end of the endoscope such that the sleeve closely fits around that endoscope; a flap hingedly attached to an edge of the proximal opening on the sleeve, wherein the flap has an adhesive strip, such that the flap folds over the proximal opening and attaches to the body with the adhesive strip to maintain the enclosed endoscope in an axially aligned position within the sleeve, wherein the enclosed endoscope can only be removed from the sleeve by tearing, cutting, or slicing the sleeve open; and a pre-formed tearable perforation on the body for removing the enclosed endoscope.

In a further embodiment of the subject invention, the tubular sidewalls may have a thickness less than 2 millimeters.

In an additional embodiment of the subject invention, the flap may comprise an exterior writable surface. In another embodiment of the subject invention, the sleeve may comprise an exterior writable surface.

In another embodiment of the subject invention, the sleeve may comprise a substantially U-shaped opening on the flap, wherein the U-shaped opening is adapted to allow an external adaptor on the endoscope to protrude from underneath the closed flap.

In a further embodiment of the subject invention, the sleeve may comprise an substantially U-shaped flap attached to an edge of the flap, wherein the U-shaped flap is adapted to allow an external adaptor on the endoscope to protrude from underneath the closed flap.

In another embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

Another embodiment of the subject invention is a non-reusable elongated sleeve sized to accommodate a single endoscope with an elongated shaft, said sleeve comprising: a thin-walled hollow tubular body with a longitudinal axis composed of an elastic plastic, wherein the body comprises a generally circular cross-section; a proximal end on the body with an proximal opening; a distal end on the body with a distal opening, wherein the proximal opening has a larger diameter than the distal opening such that the body comprises a substantially cylindrical shape that is tapered; wherein the sleeve is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the sleeve closely fits around that endoscope; a substantially flat extension hingedly attached to an edge of the proximal opening on the sleeve, wherein the extension has an adhesive strip, such that the extension folds over the proximal opening and attaches to the body with the adhesive strip to maintain the enclosed endoscope in an axially aligned position within the sleeve, wherein the enclosed endoscope can only be removed by from the sleeve tearing, cutting, or slicing the sleeve open; and a pre-formed tearable perforation on the body to remove the enclosed endoscope.

In a further embodiment of the subject invention, the walls of the body may have a thickness less than 2 millimeters.

In another embodiment of the subject invention, the substantially flat extension may comprise an exterior writable surface. In another embodiment of the subject invention, the sleeve may comprise an exterior writable surface.

In an additional embodiment of the subject invention, the sleeve may comprise a substantially U-shaped opening on the substantially flat extension, wherein the U-shaped opening is adapted to allow an external adaptor on the endoscope to protrude from underneath the closed substantially flat extension.

In another embodiment of the subject invention, the sleeve may comprise an substantially U-shaped flap attached to an edge of the substantially flat extension, wherein the U-shaped flap is adapted to allow an external adaptor on the endoscope to protrude from underneath the closed substantially flat extension.

In a further embodiment of the subject invention, the substantially flat extension may fold over the proximal opening and attach to the body with the adhesive strip such that any external adaptors connected to the enclosed endoscope may freely protrude from underneath the folded flat extension.

In an additional embodiment of the subject invention, the adhesive strip may have a protective peel off strip that is removed before attaching the substantially flat extension to the body.

The subject invention also discloses a disposable elongated protective covering for a single endoscope with an elongated shaft, said protective covering comprising: a thin-walled hollow tubular body composed of a flexible plastic, wherein the body comprises a proximal end with an proximal opening and a distal end with a distal opening, further wherein the body comprises a generally circular cross-section and a substantially frustoconical configuration wherein the proximal opening has a larger diameter than the distal opening; wherein the protective covering is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the protective covering closely fits around that endoscope; a flap hingedly attached to the protective covering proximate to the proximal opening on the protective covering, wherein the flap has an adhesive strip, such that the flap folds over the proximal opening and attaches to the body with the adhesive strip to maintain the enclosed endoscope in an axially aligned position within the protective covering, wherein the enclosed endoscope can only be removed from the protective covering by tearing, cutting, or slicing the protective covering open; and a pre-formed tearable perforation on the body to remove the enclosed endoscope.

Another embodiment of the subject invention is a non-reusable elongated protective covering sized to accommodate a single endoscope with an elongated shaft, said protective covering comprising: a thin-walled hollow tubular body with a longitudinal axis composed of an elastic plastic, wherein the body comprises a generally circular cross-section; a proximal end on the body with an proximal opening; a distal end on the body with a distal opening, wherein the proximal opening has a larger diameter than the distal opening such that the body comprises a substantially frustoconical configuration; wherein the protective covering is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the protective covering closely fits around that endoscope; a flap hingedly attached proximate to the proximal opening on the protective covering, wherein the flap has an adhesive strip, such that the flap folds over the proximal opening and attaches to the body with the adhesive strip to maintain the enclosed endoscope in an axially aligned position within the protective covering, wherein the enclosed endoscope can only be removed from the protective covering by tearing, cutting, or slicing the protective covering open; and a pre-formed tearable perforation on the body to remove the enclosed endoscope.

In a further embodiment of the subject invention, the walls of the body may have a thickness less than 2 millimeters.

In another embodiment of the subject invention, the flap may comprise an exterior writable surface. In a further embodiment of the subject invention, the protective covering may comprise an exterior writable surface.

In an additional embodiment of the subject invention, the protective covering may comprise a substantially U-shaped opening on the flap, wherein the U-shaped opening is adapted to allow an external adaptor on the endoscope to protrude from underneath the closed flap.

In another embodiment of the subject invention, the protective covering may comprise an substantially U-shaped flap attached to an edge of the flap, wherein the U-shaped flap is adapted to allow an external adaptor on the endoscope to protrude from underneath the closed flap.

In an additional embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

Another embodiment of the subject invention is a disposable single-use elongated protective covering sized to protectively cover a single endoscope with an elongated shaft, said protective covering comprising: elongated tubular sidewalls composed of a flexible plastic; a proximal end with an proximal opening; a distal end with a distal opening, wherein the protective covering comprises a generally circular cross-section and a substantially frustoconical configuration wherein the proximal opening has a larger diameter than the distal opening; wherein the proximal opening is adapted to receive the entire endoscope and the distal opening is adapted to receive only a small portion of the distal end of the endoscope such that the protective covering closely fits around that endoscope; a flap hingedly attached to an edge of the proximal opening on the protective covering, wherein the flap has an adhesive strip, such that the flap folds over the proximal opening and attaches to the body with the adhesive strip to maintain the enclosed endoscope in an axially aligned position within the protective covering, wherein the enclosed endoscope can only be removed from the protective covering by tearing, cutting, or slicing the protective covering open; and a pre-formed tearable perforation on the body for removing the enclosed endoscope.

In a further embodiment of the subject invention, the tubular sidewalls may have a thickness less than 2 millimeters.

In another embodiment of the subject invention, the flap may comprise an exterior writable surface. In another embodiment of the subject invention, the protective covering may comprise an exterior writable surface.

In a further embodiment of the subject invention, the protective covering may comprise a substantially U-shaped opening on the flap, wherein the U-shaped opening is adapted to allow an external adaptor on the endoscope to protrude from underneath the closed flap.

In another embodiment of the subject invention, the protective covering may comprise an substantially U-shaped flap attached to an edge of the flap, wherein the U-shaped flap is adapted to allow an external adaptor on the endoscope to protrude from underneath the closed flap.

In an additional embodiment of the subject invention, the flap may fold over the proximal opening and attach to the body with the adhesive strip such that any external adaptors connected to the enclosed endoscope may freely protrude from underneath the folded flap.

In a further embodiment of the subject invention, the adhesive strip may have a protective peel off strip that is removed before attaching the flap to the body.

Another embodiment of the subject invention is a non-reusable elongated protective covering sized to accommodate a single endoscope with an elongated shaft, said protective covering comprising: a thin-walled hollow tubular body with a longitudinal axis composed of an elastic plastic, wherein the body comprises a generally circular cross-section; a proximal end on the body with an proximal opening; a distal end on the body with a distal opening, wherein the proximal opening has a larger diameter than the distal opening such that the body comprises a substantially cylindrical shape that is tapered; wherein the protective covering is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the protective covering closely fits around that endoscope; a substantially flat extension hingedly attached to an edge of the proximal opening on the protective covering, wherein the extension has an adhesive strip, such that the extension folds over the proximal opening and attaches to the body with the adhesive strip to maintain the enclosed endoscope in an axially aligned position within the protective covering, wherein the enclosed endoscope can only be removed from the protective covering by tearing, cutting, or slicing the protective covering open; and a pre-formed tearable perforation on the body to remove the enclosed endoscope.

In a further embodiment of the subject invention, the walls of the body may have a thickness less than 2 millimeters.

In another embodiment of the subject invention, the substantially flat extension may comprise an exterior writable surface. In a further embodiment of the subject invention, the protective covering may comprise an exterior writable surface.

In an additional embodiment of the subject invention, the protective covering may comprise a substantially U-shaped opening on the substantially flat extension, wherein the U-shaped opening is adapted to allow an external adaptor on the endoscope to protrude from underneath the closed substantially flat extension.

In another embodiment of the subject invention, the protective covering may comprise an substantially U-shaped flap attached to an edge of the substantially flat extension, wherein the U-shaped flap is adapted to allow an external adaptor on the endoscope to protrude from underneath the closed substantially flat extension.

In an additional embodiment of the subject invention, the substantially flat extension may fold over the proximal opening and attach to the body with the adhesive strip such that any external adaptors connected to the enclosed endoscope may freely protrude from underneath the folded flat extension.

In a further embodiment of the subject invention, the adhesive strip may have a protective peel off strip that is removed before attaching the substantially flat extension to the body.

In embodiments of the subject invention, the sheath, sleeves and protective coverings may comprise a plastic that is substantially transparent.

In further embodiments of the subject invention, the sheath, sleeves and protective coverings may comprise a plurality of colors.

In additional embodiments of the subject invention, the sheath, sleeves and protective coverings may comprise a thermoplastic polymer. In further embodiments of the subject invention, the sheath, sleeves and protective coverings may comprise a polyethylene. In embodiments of the subject invention, the sheath, sleeves and protective coverings may comprise a pigmented thermoplastic polymer.

In further embodiments of the subject invention, wherein the sheaths, sleeves and protective coverings may comprise a length of 30 to 80 inches. In additional embodiments of the subject invention, the proximal opening may comprise a circumference of 10 to 16 inches. In further embodiments of the subject invention, the distal opening may comprise a circumference of 2 to 7 inches.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto. These together with other objects of the invention, along with the various features of novelty, which characterize the invention, are pointed out with particularity in the claims annexed to and forming a part of this disclosure.

### BRIEF DESCRIPTION OF THE DRAWINGS

Advantages of the present invention will be apparent from the following detailed description of exemplary embodiments thereof, which description should be considered in conjunction with the accompanying drawings:

FIG. 1 illustrates a front view of a disposable flexible endoscope cover of the subject invention in the open position, with the closing flap raised.

FIG. 2 illustrates a front view of a disposable flexible endoscope cover of the subject invention in the closed position with the closing flap folded over and adhered to the cover.

FIG. 3 illustrates a front view of a disposable flexible endoscope cover of the subject invention in the open position with an endoscope being inserted.

FIG. 4 illustrates a rear view of a disposable flexible endoscope cover of the subject invention in the open position with an endoscope fully inserted into the cover.

FIG. 5 illustrates a front view of a disposable flexible endoscope cover of the subject invention in the closed position with an endoscope fully inserted into the cover.

FIG. 6 illustrates a front view of a disposable flexible endoscope cover of the subject invention in the re-opened position with an endoscope being removed from the torn cover.

FIG. 7 illustrates a front view of another embodiment of a disposable flexible endoscope cover of the subject invention in the open position, with the closing flap raised in which the closing flap has a U-shaped opening.

FIG. 8 illustrates a front view of another embodiment of a disposable flexible endoscope cover of the subject invention in the closed position with the closing flap folded over and adhered to the cover in which the closing flap has a U-shaped opening.

FIG. 9 illustrates a front view of a further embodiment of a disposable flexible endoscope cover of the subject invention in the open position, with the closing flap raised in which the closing flap has a U-shaped flap.

FIG. 10 illustrates a front view of a further embodiment of a disposable flexible endoscope cover of the subject invention in the closed position with the closing flap folded over and adhered to the cover in which the closing flap has a U-shaped flap.

### DETAILED DESCRIPTION OF EMBODIMENTS

The following will describe, in detail, several embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

For a conceptual understanding of the invention and its operational advantages, refer to the accompanying drawings and descriptive matter in which there are preferred embodiments of the invention illustrated. Other features and advantages of the present invention will become apparent from the following description of the preferred embodiment(s), taken in conjunction with the accompanying drawings, which by way of example; illustrate the principles of the invention.

The subject invention is a system of disposable, one-use protective covers (sheaths or sleeves) for labeling de-contaminated and cleaned flexible endoscopes with elongated shafts.

After a flexible endoscope is used in a surgical procedure, the endoscope is disinfected and de-contaminated. These “cleaned” endoscopes are then usually kept by medical care providers in separate areas to distinguish them from contaminated or “dirty” endoscopes that have not yet been cleaned. During the fast pace of providing medical care, medical care professionals may inadvertently mistake a “dirty” endoscope for a “clean” endoscope, resulting in a greater risk of patient to patient cross contamination.

The subject invention is a system of translucent one-use, disposable coverings (or sleeves), preferably made of lightweight plastics, with shapes that are specially contoured to encapsulate multiple types of flexible endoscopes.

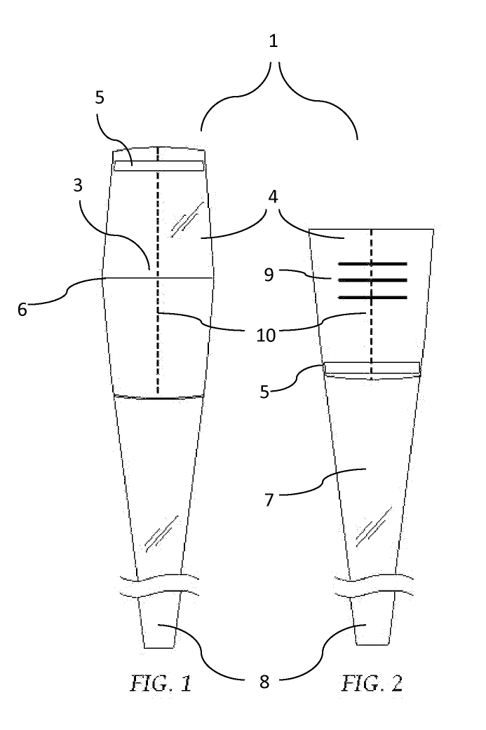

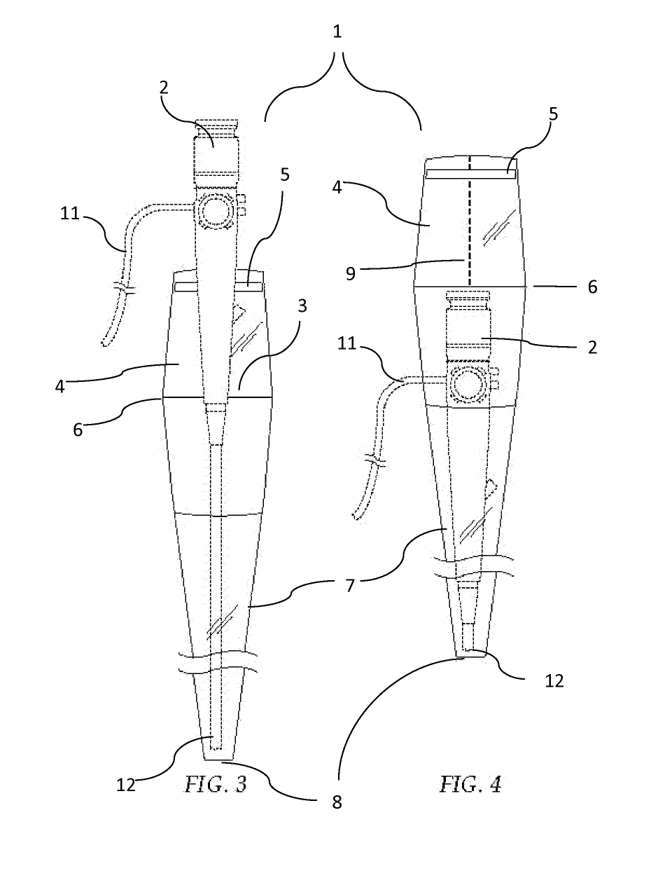

As illustrated in FIGS. 1 through 10, a disposable cover 1 of the subject system contains a thinned-walled substantially tubular body composed of a flexible, elastic plastic. In one embodiment of the subject invention, the disposable covers of the subject invention may be a lightweight clear thin wall made from 1.5 mm thick polyethylene material.

The cover 1 contains an open proximal end 3 and an open distal end 8. The cover 1, the proximal end 3, and the distal end 8 each have generally circular cross-sectional shapes. The diameter of open proximal end 3 is larger than the diameter of open distal end 8, and the diameter of the cover 1 smoothly tapers as it extends from the proximal end 3 to distal end 8 to form a substantially frustoconical configuration.

Proximal end 3 has a flap 4 hingedly attached proximate to or on the edge of the opening of proximal end 3. This configuration allows a flexible endoscope 2 to be inserted into cover 1. FIGS. 1, 3 and 4 illustrate the cover 1 in the “open” position with flap 4 raised. A distal tip 12 of clean endoscope 2 is inserted into cover 1 in the open position; lower tip 12 first, through the opening of proximal end 3. As the distal tip 12 of the endoscope 2 passes through lower portion 7 of the cover 1, tip 12 passes through the opening of distal end 8. The open-ended distal end 8 on cover 1 is only large enough to allow the distal tip 12 of the endoscope 2 to pass through and keeps the rest of endoscope 2 enclosed within cover 1. This open-ended distal end 8 on cover 1 allows endoscopes with different size tips 12 to be inserted into the cover 1 without the need to remove or adjust the tip 12.

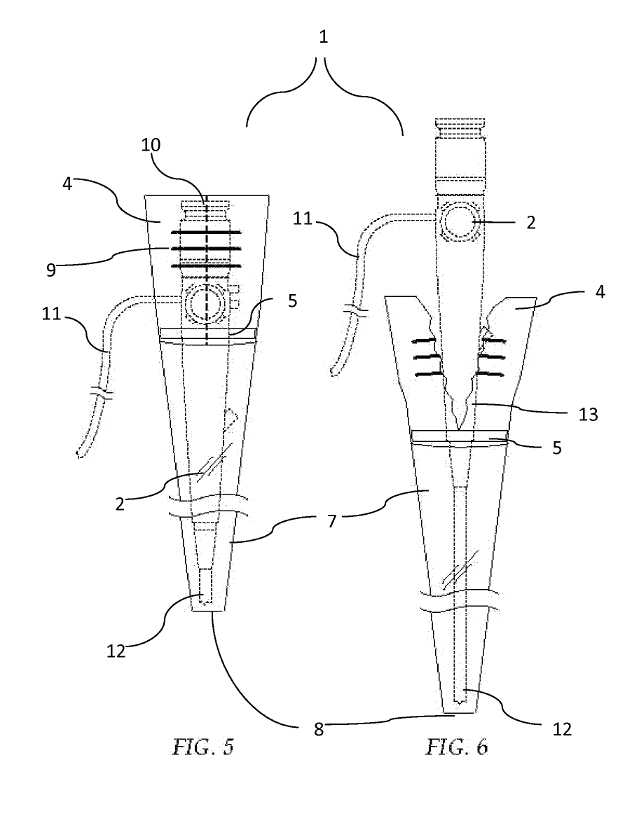

Once the endoscope 2 is fully inserted into the cover 1, flap 4 is folded down over the opening of proximal end 3 along pre-folded crease 6 on the cover 1. Flap 4 is folded completely over the contained endoscope 2 until an adhesive strip 5 on outermost end of flap 4 may be used to close the opening of proximal end 3. The adhesive strip 5 may contain a protective strip that is removed before adhering to the cover 1. This adhesive strip 5 seals the flexible endoscope 2 within the cover 1 and prevents the cover 1 from slipping. As shown in FIGS. 2 and 5, once the cover is in the “closed” position, flap 4 covers the top of endoscope 2. In this embodiment, the enclosed endoscope 2 can only be removed from cover 1 by ripping, tearing, cutting, slicing or otherwise breaking the wall of cover 1. Once cover 1 is ripped, torn, cut, sliced or broken, it cannot be re-sealed. Any medical provider that views a cover 1 that is ripped, torn, cut, sliced or broken, he or she can quickly deduce that the endoscope is not clean and is not safe for use. Therefore, this system reduces the chances of cross-contamination with used endoscopes.

Flap 4 closes over the endoscope 2 such that any external tubing 11 or light source adaptor connected to the endoscope 2 can freely protrude from underneath the closed flap 4. This flexible closing flap 4 allows endoscopes with different size tubings 11 or adaptors to be inserted into the cover 1 without the need to remove or adjust the tubing 11 or adaptor.

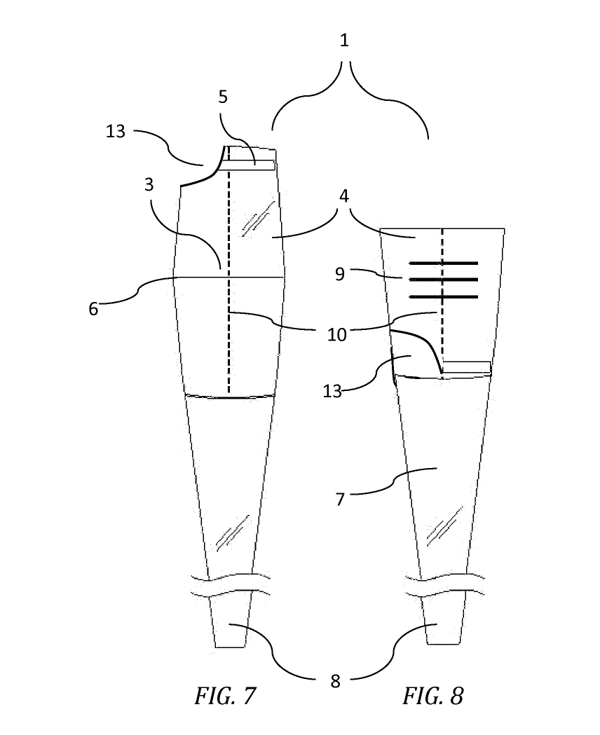

FIGS. 7 and 8 illustrate another embodiment of the subject invention. In this embodiment, the cover 1 may include a substantially U-shaped opening 13 on flap 4 to allow any external tubing 11 or light source adaptor to freely protrude from underneath the closed flap. In embodiments of the subject invention, this U-shaped opening 13 may be 1.5 inches in width by 1.5 inches in height.

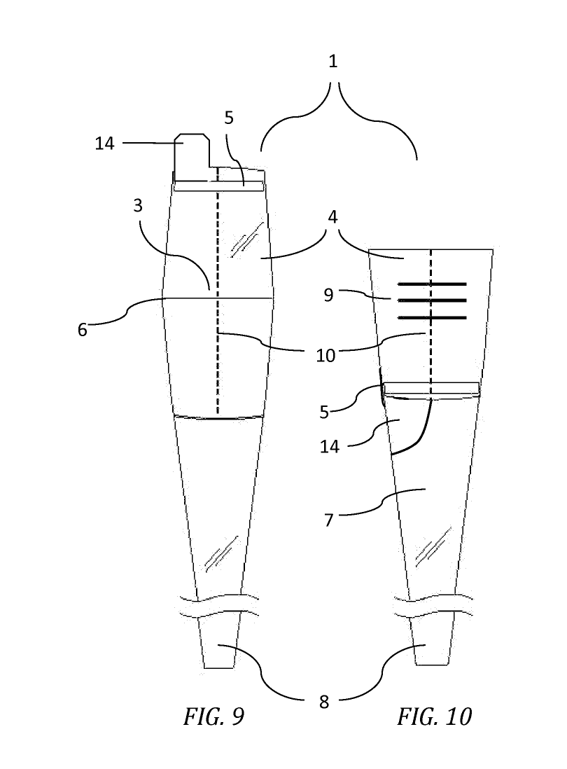

FIGS. 9 and 10 illustrate a further embodiment of the subject invention. In this embodiment, the cover 1 may include a substantially U-shaped flap 14 on flap 4 to allow any external tubing 11 or light source adaptor to freely protrude from underneath the closed flap. In embodiments of the subject invention, this U-shaped flap 14 may be 1.5 inches in width by 1.5 inches in height.

In one embodiment of the subject invention, the back of flap 4 may contain writable surfaces for medical care providers to write various indicia 9, including, but not limited to: date of cleaning, location, and initials of the last user. After the disposable cover is sealed, a user may write on these areas to identify the last de-contamination of the enclosed endoscope 2.

The sealed and labeled endoscopes may now be stored in common endoscope storage cabinets. The disposable cover will also allow flexible endoscopes to be stored in a vertical hanging position in existing endoscope closets or hung to be transported on a cart.

In one embodiment of the subject invention, the disposable covers of the subject invention may be manufactured in different dimensions to specifically contour to the shapes of various sized endoscopes. These disposable covers may contain different colored strips running the length of the cover (not shown) to identify the particular size.

For example, in one embodiment of the subject application, a disposable cover 1 for a Colonscope may have a length, from proximal end 3 to distal end 8, of 76 inches, with a top circumference of 14 inches around the opening of proximal end 3 and a bottom circumference of 6 inches around the opening of distal end 8.

In another embodiment of the subject application, a disposable cover 1 for a Gastroscope may have a length, from proximal end 3 to distal end 8, of 56 inches, with a top circumference of 14 inches around the opening of proximal end 3 and a bottom circumference of 6 inches around the opening of distal end 8.

In a further embodiment of the subject application, a disposable cover 1 for a Bronchoscope may have a length, from proximal end 3 to distal end 8, of 34 inches, with a top circumference of 12 inches around the opening of proximal end 3 and a bottom circumference of 4 inches around the opening of distal end 8.

A medical care provider that needs an endoscope for surgery will grab the clean, sealed endoscope. In this embodiment, the enclosed endoscope 2 can only be removed from cover 1 by ripping, tearing, cutting, slicing or otherwise breaking the wall of cover 1. Once cover 1 is ripped, torn, cut, sliced or broken, it cannot be re-sealed. Any medical provider that views a cover 1 that is ripped, torn, cut, sliced or broken, he or she can quickly deduce that the endoscope is not clean and is not safe for use. Therefore, this system reduces the chances of cross-contamination with used endoscopes.

In one embodiment of the subject invention, flap 4 contains a pre-formed perforation 10 that allows a user to easily tear open cover 1 and retrieve the endoscope 2. As shown in FIG. 6, perforation 10 allows a user to tear open cover 1 and easily remove clean endoscope 2 for use. In other embodiments of the subject invention, the perforations may be along the side or back of cover 1. Once a cover 1 is removed, it cannot be resealed or re-used, thus preventing cross-contamination.

This process for cover 1 removal is easy and quick and will not interfere with endoscope usage in emergencies.

The subject system of disposable covers provides protection and assurance endoscope cleanliness before each use. This system reduces the risk of patient to patient cross-contamination by clearly labeling cleaned endoscopes for easy identification by medical care professionals.

What is claimed is:

1. A system for organizing de-contaminated and clean endoscopes, comprising:
cleaning and de-contaminating an endoscope;
selecting a non-reusable elongated sheath based on the type of endoscope to be enclosed from a plurality of non-reusable elongated sheaths, wherein each sheath from the plurality of sheaths closely fits a different type of endoscope, further wherein each sheath comprises indicia to indicate the type of endoscope that the sheath closely fits,
wherein each sheath comprises a thin-walled hollow tubular body composed of a flexible plastic, a proximal end with an proximal opening, a distal end with a distal opening,
wherein each sheath comprises a generally circular cross-section and a substantially frustoconical configuration such that the proximal opening has a larger diameter than the distal opening;
placing the endoscope into the selected sheath by inserting the distal end of the endoscope through the proximal opening, wherein the proximal end of the sheath is adapted to allow the endoscope to pass through it;
passing the distal end of the endoscope through the hollow tubular body;
passing a small portion of the distal end of the endoscope through the distal opening such that the sheath closely fits around that endoscope, wherein the distal opening of the sheath is adapted to only allow the small portion of the distal end of the endoscope to pass through;
folding a flap that is hingedly attached to the sheath proximate to the proximal opening over the proximal opening, wherein the flap has an adhesive strip;
attaching the folded flap to the body with the adhesive strip to maintain the enclosed endoscope in an axially aligned position within the sheath, wherein the enclosed endoscope can only be removed from the sheath by breaking the thin wall of the sheath;
placing the sheathed endoscope into a location for clean and de-contaminated endoscopes designated by a user;
removing the sheathed endoscope for use by breaking the thin wall of the sheath and removing the enclosed endoscope; and
discarding the used sheath.

2. The system of claim 1, wherein the indicia to indicate the type of endoscope that each sheath closely fits comprises a color-code.

3. The system of claim 1, wherein the indicia to indicate the type of endoscope that each sheath closely fits comprises a printed label.

4. The system of claim 1, wherein the walls of the body comprise a thickness less than 2 millimeters.

5. The system of claim 1, wherein the flap comprises an exterior writable surface.

6. The system of claim 1, wherein the sheath comprises an exterior writable surface.

7. The system of claim 1, wherein the sheath further comprises a substantially U-shaped opening on the flap, wherein the U-shaped opening is adapted to allow an external adaptor on the endoscope to protrude from underneath the closed flap.

8. The system of claim 1, wherein the sheath further comprises an substantially U-shaped flap attached to an edge of the flap, wherein the U-shaped flap is adapted to allow an external adaptor on the endoscope to protrude from underneath the closed flap.

9. The system of claim 1, wherein the flap folds over the proximal opening and attaches to the body with the adhesive strip such that any external adaptors connected to the enclosed endoscope freely protrude from underneath the folded flap.

10. The system of claim 1, wherein the adhesive strip comprises a protective peel off strip that is removed before attaching the flap to the body.

11. The system of claim 1, wherein the sheath comprises a plastic that is substantially transparent.

12. The system of claim 1, wherein the sheath comprises a thermoplastic polymer.

13. The system of claim 1, wherein the sheath comprises a polyethylene.

14. The system of claim 1, wherein the sheath comprises a pigmented thermoplastic polymer.

15. The system of claim 1, wherein the sheath comprises a length of 30 to 80 inches.

16. The system of claim 1, wherein the proximal opening comprises a circumference of 10 to 16 inches.

17. The system of claim 1, wherein the distal opening comprises a circumference of 2 to 7 inches.

18. A disposable single-use elongated sheath adapted for covering a single endoscope with an elongated shaft, said sheath comprising:
a thin-walled hollow tubular body composed of a flexible plastic, wherein the body comprises a proximal end with an proximal opening and a distal end with a distal opening,
further wherein the body comprises a generally circular cross-section and a substantially frustoconical configuration wherein the proximal opening has a larger diameter than the distal opening;
wherein the sheath is adapted to cover the endoscope by receiving the distal end of the endoscope through the proximal opening, allowing the distal end of the endoscope to pass through the hollow tubular body and allowing only a small portion of the distal end of the endoscope to pass through the distal opening such that the sheath closely fits around that endoscope;
a flap hingedly attached to the sheath proximate to the proximal opening on the sheath, wherein the flap has an adhesive strip, such that the flap folds over the proximal opening and attaches to the body with the adhesive strip to maintain the enclosed endoscope in an axially aligned position within the sheath,
wherein the enclosed endoscope can only be removed from the sheath by breaking the thin wall of the sheath.

19. The system of claim 1, wherein the walls of the body comprises a thickness less than 2 millimeters.

20. The system of claim 1, wherein the sheath comprises an exterior writable surface.
